(19) United States
(12) Patent Application Publication
(10) Pub. No.: US 2007/0135860 A1
(43) Pub. Date: Jun. 14, 2007

(54) Device for treating patients by brain stimulation, electronic component and use of the device and electronic component in medicine and medical treatment method

(75) Inventor: Peter Tass, Titz (DE)

Correspondence Address: STAAS & HALSEY LLP, SUITE 700, 1201 NEW YORK AVENUE, N.W., WASHINGTON, DC, 20005, US

(73) Assignee: FORSCHUNGSZENTRUM JULICH GmBH, Julich (DE)

(21) Appl. No.: 11/603,293

(22) Filed: Nov. 22, 2006

Related U.S. Application Data
(63) Continuation of application No. PCT/DE05/00747, filed on Apr. 23, 2005.

(30) Foreign Application Priority Data
May 24, 2004 (DE) 10 2004 025 825.2

(51) Int. Cl. A61N 1/00

(52) U.S. Cl. 607/45

(57) ABSTRACT

A device for treating patients by brain stimulation and related electronic component and use of the device and of the electronic component in a medical treatment method. To achieve the brain stimulation result, a device includes at least one electrode for stimulating a brain region, at least one sensor for measuring an electrical signal, a control system which can detect the occurrence of a pathological feature of the electrical signal which was measured by the sensor and, when the pathological feature occurs, delivers at least one component from a group of stimulus sequences (a) through (d): (a) a short high-frequency pulse train, (b) a resetting short high-frequency pulse train followed by a further desynchronizing short high-frequency pulse train, (c) a resetting low-frequency sequence of a high-frequency pulse train followed by a desynchronizing high-frequency pulse train, or (d) a resetting single pulse followed by a short desynchronizing high-frequency pulse train to the electrode.

### CROSS-REFERENCE TO RELATED APPLICATIONS

This application is a continuing application, filed under 35 U.S.C. §111(a), of International Application PCT/DE2005/000747, filed on Apr. 23, 2005, it being further noted that priority is based upon German Patent Application 10 2004 025 825.2, filed on May 24, 2004, the contents of which are incorporated herein by reference.

### BACKGROUND OF THE INVENTION

The invention relates to a device for treating patients by brain stimulation and related electronic component and use of the device and of the electronic component in medicine and a medical treatment method.

### SUMMARY OF THE INVENTION

Additional aspects and/or advantages of the invention will be set forth in part in the description which follows and, in part, will be apparent from the description, or may be learned by practice of the invention.

In patients with neurological or psychiatric diseases such as, for example, Parkinson's disease, essential tremor, dystonia or compulsive diseases, nerve cell populations are pathologically active, for example excessively synchronous, in defined areas of the brain, e.g. the thalamus and the basal ganglia. In this case, a large number of neurons synchronously form action potentials, that is to say the neurons involved fire excessively synchronously. In a healthy patient, the neurons fire qualitatively differently in these brain regions, for example in uncorrelated manner.

In Parkinson's disease, the pathologically synchronous activity changes the neural activity in areas of the cerebral cortex such as, for example, in the primary motor cortex, for example by forcing their rhythm onto these, so that finally the muscles controlled by these areas develop pathological activity, e.g. a rhythmic trembling.

In patients which can no longer be treated by medicaments, a depth electrode is implanted depending on whether the disease occurs unilaterally or bilaterally. In this arrangement a cable leads under the skin from the head to the so-called generator which comprises a control device with a battery and is implanted, for example, in the area of the clavicle under the skin. Via the depth electrodes, continuous stimulation is carried out with a high-frequency periodic sequence (at a frequency of >100 Hz) of individual stimuli, for example at rectangular pulses (pulse train). It is the aim of this method to suppress the firing of the neurons in the target areas. This standard depth simulation acts like a reversible lesion—that is to say like a reversible elimination of the tissue. The active mechanisms, i.e. how precisely standard stimulation works, has not yet been explained adequately.

However, the method hitherto used has some disadvantages. Thus, the energy consumption achieved with the continuous stimulation is very high so that the generator and its battery frequently have to be exchanged operatively after only approximately one to three years.

It is particularly disadvantageous, however, that the continuous high-frequency stimulation, as an unphysiological, that is to say unnatural input in the area of the brain, for example the thalamus or the basal ganglia, can lead to an adaptation of the nerve cell populations affected in the course of a few years. To achieve the same stimulation success, a higher stimulus amplitude must then be used for simulating due to this adaptation. The greater the stimulus amplitude, the greater the probability that, due to the stimulation of neighboring areas, side effects such as dysarthria (speech disturbances), dysesthesia (in some cases very painful abnormal sensations), cerebellar ataxia (inability to stand without help), depression or schizophrenic symptoms etc. These side effects cannot be tolerated by the patient. In these cases, the treatment, therefore, loses its effectiveness after a few years.

German patent application 102 11 766.7 by the applicant discloses a device for treating patients by means of brain stimulation in which, in order to desynchronize the neural activity when a control system detects a pathological feature, either a) a high-frequency pulse train followed by a single pulse or b) a low-frequency pulse train followed by a single pulse or c) a high-frequency pulse train are applied.

The disadvantage of this method described in application 102 11 766.7 is that the single pulses are not always optimally effective. In the case of inadequate effectiveness, the amplitude of the single stimuli must be selected to be relatively high so that side effects can occur—e.g. due to propagation of the stimulation current to adjacent brain regions.

It is the object of the invention, therefore, to create a device which provides for more efficient treatment than with the device according to DE 102 11 766.7, in which symptoms of the respective disease are reduced or completely eliminated. In this device, it is intended not only simply to suppress the activity of the nerve cell populations affected but to bring it closer to the healthy state of functioning. Furthermore, the side effects such as, for example, the dysarthria, dysesthesia, cerebellar ataxia, depression or schizophrenic symptoms etc., which occur in accordance with the methods according to the prior art, are to be eliminated or at least reduced. In comparison with the device and the method according to application DE 102 11 766.7, a method and a device are to be created which manage with lower stimulus amplitudes, particularly in order to reduce or eliminate side effects for the patient.

Based on the preamble of claim 1, the object is achieved, according to the invention, by means of the features specified in the characterizing clause of claim 1.

The device according to the invention now makes it possible to treat patients without any adaptation to the unphysiological continuous stimulus occurring, the abovementioned side effects being reduced or eliminated. By using the device according to the invention, the battery or current consumption can be additionally drastically reduced which is why the batteries need to be exchanged or charged up less frequently. The device according to the invention can operate with lower stimulus amplitude and leads to an improved therapeutic effect in comparison with the device from DE 102 11 766.7.

Advantageous refinements of the invention are specified in the subclaims.

### BRIEF DESCRIPTION OF THE DRAWINGS

The drawings show an exemplary embodiment of the device according to the invention and stimulus patterns according to the invention.

FIG. 1 shows a block diagram of the device,

FIG. 2 shows exemplary pulse sequences according to the invention.

### DETAILED DESCRIPTION OF THE PREFERRED EMBODIMENTS

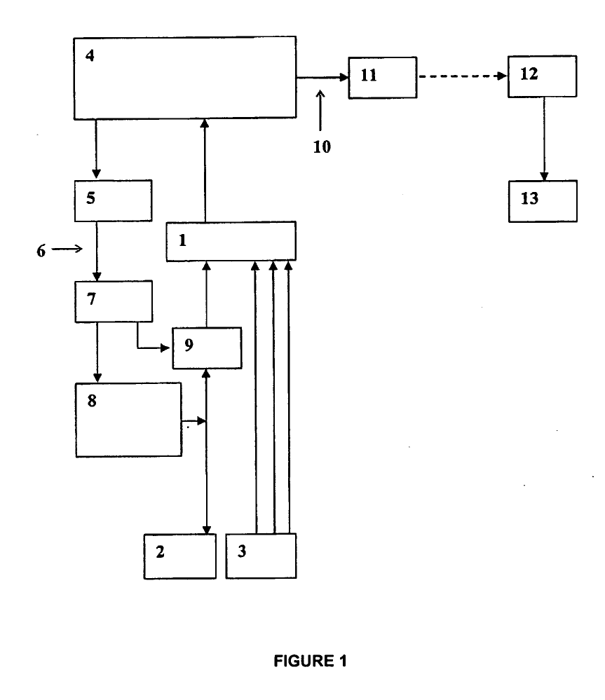

The device according to the invention, shown in FIG. 1, comprises an isolating amplifier (1), to which at least one electrode (2) and sensors (3) for detecting physiological measurement signals are connected. The isolating amplifier is also connected to a unit (4) for signal processing and control which is connected to an optical transmitter for the stimulation (5). The optical transmitter (5) is connected by optical waveguides (6) to an optical receiver (7) which is connected to a stimulator unit (8) for signal generation. The stimulator unit (8) for signal generation is connected to the electrode (2). At the input area of the electrode (2) into the isolating amplifier (1), a relay (9) or transistor is located. The unit (4) is connected via a line (10) to a telemetry transmitter (11) which is connected to a telemetry receiver (12) which is located outside the device to be implanted and to which a means for displaying, processing and storing the data (13) is connected.

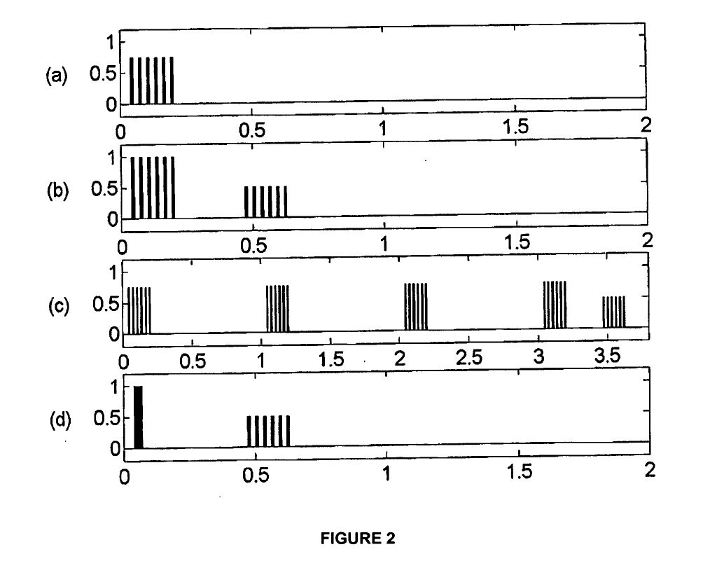

FIG. 2 shows by way of example the stimulus patterns according to the invention. In FIGS. 2a to 2d, the ordinate corresponds to the current intensity and the abscissa corresponds to time, both being represented in arbitrary units. In all figures, a single pulse is shown diagrammatically as rectangular block.

FIG. 2a shows a single high-frequency pulse train which consists of six single pulses.

FIG. 2b shows a resetting high-frequency pulse train which is followed by a desynchronizing high-frequency pulse train.

FIG. 2c shows a low-frequency resetting sequence of high-frequency pulse trains which is followed by a desynchronizing high-frequency pulse train.

FIG. 2d shows a resetting single pulse followed by a desynchronizing high-frequency pulse train.

The sensors (3) used can be, for example, epicortical electrodes, depth electrodes, brain electrodes or peripheral electrodes.

The electrode (2) consists of at least two wires, at the ends of which a potential difference is applied for the purpose of stimulation. The electrode (2) is a means for stimulus application. In the wider sense, it can also be a means for measuring physiological signals. They can be macro or microelectrodes. In addition, but not mandatorily, a potential difference can be measured via the electrode (2) in order to detect a pathological activity. In a further embodiment, the electrode (2) can also consist of only a single wire. In this case, a potential difference is applied between the end of this wire, on the one hand, and a metallic counterpiece, on the other hand, for the purpose of stimulation. The metallic counterpiece can be, for example, a housing of the device or of a part thereof or any other electrode or another metallic object which is connected to the stimulator unit (8) analogously to the wire of the electrode (2). In a further embodiment, the electrode (2) can also consist of more than two single wires which can be used both for determining a measurement signal in the brain and for the stimulation. For example, four wires can be accommodated in a conductor cable and a potential difference can be applied or measured between different ends. As a result, the size of the target area derived or stimulated can be varied. The number of wires of which the electrode is constructed is limited towards upper values only by the associated thickness of the cable to be introduced into the brain so that as little brain material as possible will be damaged. Commercially available electrodes comprise four wires but five, six or more wires or only three wires can also be comprised. Suitable electrodes are known to the expert and not restricted to the electrodes listed by way of example.

In the case where the electrode (2) comprises more than two wires, at least two of these wires can also act as sensor (3) so that, in this special case, this is an embodiment in which the electrode (2) and the sensor (3) are combined in a single component. The wires of the electrode (2) can have different lengths so that they can penetrate into different brain depths. If the electrode (2) consists of n wires, a stimulation can be effected via at least one pair of wires, any subcombination of wires being possible when forming the pair. Apart from this component, sensors (3) not constructionally combined with the electrode (2) can be present.

The unit for signal processing and control 4 comprises means for univariate and/or bivariate data processing as is described, for example, in “Detection of n:m Phase Locking from Noisy Data: Application to Magnetoencephalography”, by P. Tass et al., in Physical Review Letters, 81,3291 (1998).

According to the invention, the device is equipped with means which detect the signals of the electrode (2) or of the sensors (3) as pathological and, in the case of the presence of a pathological pattern, deliver via the electrode (2) stimuli which have the effect that the pathological neural activity is either temporarily suppressed or modified in such a manner that it becomes closer to the natural physiological activity. The pathological activity differs from the healthy activity by a characteristic change in its pattern and/or its amplitude which are known to the expert and which can be detected by known methods.

The means for detecting the pathological pattern are a computer, which processes the measured signals of the electrode (2) and/or of the sensor (3) and compares them with data stored in the computer. The computer has a data medium which stores data which have been determined as part of a calibration procedure. For example, these data can be determined by varying the stimulation parameters systematically in a series of test stimuli and determining the success of the stimulation via the electrode (2) and/or the sensor (3) by means of the control unit (4). The determination can be made by uni- and/or bi- and/or multivariate data analysis for characterizing the frequency characteristics and the interaction (e.g. coherence, phase synchronization, directionality and stimulus/response relation) as has been disclosed, for example, in P. A. Tass: “Phase resetting in Medicine and Biology, Stochastic Modelling and Data Analysis.” Springer Verlag, Berlin 1999.

The device according to the invention, therefore, comprises a computer which contains a data medium which carries the data of the disease pattern, compares it with the measurement data and, in the case of the occurrence of pathological activity, delivers a stimulus signal to the electrode (2) so that the brain tissue is stimulated. The data of the disease pattern stored in the data medium can be either person-related optimal stimulation parameters determined by calibration or a data pattern which has been determined from a group of patients and represents optimal stimulation parameters occurring typically. The computer recognizes the pathological pattern and/or the pathological amplitude.

The control unit (4) can comprise, for example, a chip or another electronic device with comparable computing power.

The control unit (4) preferably controls the electrode (2) in the following manner. The control data are forwarded by the control unit (4) to an optical transmitter for the stimulation (5) which drives the optical receiver (7) via the optical waveguide (6). The optical coupling of control signals into the optical receiver (7) results in DC-decoupling of the stimulation control from the electrode (2) which means that any injection of interfering signals from the unit for signal processing and control (4) into the electrode (2) is prevented. The optical receiver (7) to be considered is, for example, a photocell. The optical receiver (7) forwards the signals input via the optical transmitter for the stimulation (5) to the stimulator unit (8). Via the stimulator unit (8), selective stimuli are then forwarded via the electrodes (2) to the target region in the brain. In the case where measurements are also made via the electrode (2), a relay (9) is also activated from the optical transmitter for the stimulation (5) via the optical receiver (7) which prevents the injection of interfering signals. The relay (9) or the transistor ensures that the neural activity can be measured again immediately after each stimulus without the isolating amplifier being overdriven. The DC decoupling does not necessarily have to be effected by coupling in the control signals optically and other alternative control systems can also be used, instead. These can be, for example, acoustic couplings, for example in the ultrasonic range. Interference-free control can also be implemented, for example, with the aid of suitable analog or digital filters.

Furthermore, the device according to the invention is preferably connected to means for displaying and processing the signals and for saving the data (13) via the telemetry receiver (12). The unit (13) can have the above-mentioned methods for uni- and/or bi- and/or multivariate data analysis.

Furthermore, the device according to the invention can be connected via the telemetry receiver (13) to an additional reference database, in order to, for example, accelerate the calibration process.

In neurosurgery, two types of stimulation are typically used: 1. continuous high-frequency stimulation (for suppressing neural activity) and 2. low-frequency stimulation (for reinforcing or exciting neural activity). The frequency of the continuous high-frequency stimulation is typically greater than 100 Hz, e.g. 130 Hz. The frequency of the continuous low-frequency stimulation, in contrast, has values about 2 Hz to 30 Hz.

In the device according to the invention, in contrast, novel forms of stimulus are used which influence the phase dynamics and the extent of the synchronization of neural rhythmic activity in a particularly efficient manner. It has been found surprisingly that the more complex stimulus sequences described below and composed of short high-frequency pulse trains bring the pathologically synchronous activity close to the natural non-pathological activity, or completely match it, in a particularly effective manner.

The device according to the invention is used for measuring the pathological neural activity via an electrode (2) such as a) a brain electrode, e.g. a depth electrode, b) an epicortical electrode or via c) a muscle electrode and is used as feedback signal, that is to say control signal, for a demand-controlled stimulation. The feedback signal from the sensor (3) is transmitted by a line to the isolating amplifier (1). As an alternative, the feedback signal can also be transmitted telemetrically without using an isolating amplifier. In the case of telemetric transmission, the sensor (3) is connected to an amplifier via a cable. The amplifier is connected to a telemetry transmitter via a cable. In this case, the sensor (3) and amplifier and telemetry transmitter are implanted, for example, in the area of an extremity affected whereas the telemetry receiver is connected to the control unit (4) via a cable. This means that the activity is measured and the measurement signal is used as a trigger for a demand-controlled stimulation.

The following various possibilities exist for measuring the neural activity:
I. Measurement via the brain electrode a) (electrode (2)) which in this case also handles the function of a sensor (3), which is also used for stimulating. If the electrode (2) consists of more than three wires, at least two of these wires can act as sensor (3), these wires not being used for stimulating in this case.
II. Measuring the neural activity from deeper areas of the brain such as thalamus or basal ganglia via the depth electrode a′) (sensor (3)) which is not used for stimulating. In this case, a further depth electrode a′) is used as sensor (3) in addition to the depth electrode a) acting as electrode (2).
III. Measuring neural activity which comes from the cerebral cortex, either via an implanted electrode b) or preferably an atraumatic epicortical electrode b) (sensor (3)), i.e. an electrode which rests on the brain is fixed but not penetrate the tissue and in this manner derives a local electroencephalogram of an affected area of the cerebral cortex, e.g. the primary motoric cortex.
IV. In patients who primarily suffer from a tremor, muscular activity can also be measured by electrodes c) (sensor (3), preferably telemetrically connected to the control unit (4)) in the area of the muscles affected.

In principle, the pathological neural activity can also occur in different neuron populations. For this reason, a number of signals measured via electrode (2) and/or sensors (3) can also be used for controlling the stimulation. Whenever a pathological feature of the activity is detected in at least one of the neuron populations, a stimulus is triggered.

The electrode (2) can also handle the function of a sensor (3). This makes it possible to derive the activity of the neuron population at the point of treatment of the electrode (2).

The measurement signal or the measurement signals is or are used as feedback signals. This means that stimulation occurs in dependence on the activity detected by the measurement signal. Whenever a pathological feature of the neural activity, that is to say pathologically increased amplitude or pathologically increased pronounced activity pattern) occurs and/or increases, stimulation is applied.

According to the invention, stimulation is thus applied when pathologically synchronized nerve cell activity is present in the target area (derived via electrode (2)) (e.g. in areas of the thalamus in Parkinson's disease) or in another area or muscle relevant to the disease (derived via sensors (3)). This is determined, for example, by the signals measured via electrode (2) and/or sensors (3) being band-pass filtered in the frequency range which is characteristic of the pathological activity. As soon as a band-pass-filtered measurement signal exceeds a threshold value, determined as part of the calibration procedure, the next control pulse is forwarded via the control unit (4) to the optical transmitter (5) which produces the stimuli generated via the electrode (2) via the optical waveguide (6) and the optical receiver (7). The aim is not simply to suppress the firing of the neurons as in standard continuous stimulation. Instead, it is only intended to eliminate the pathologically increased synchronization of the nerve cells as required. That is to say the nerve cell populations in the target area are desynchronized, still remaining active, that is to say forming action potentials. By this means, the nerve cells affected are to be brought closer to their physiological state, that is to say firing in an uncorrelated manner, instead of the activity simply being suppressed completely. For this purpose, a number of different desynchronizing methods can be used which are based on the principle of “stochastic phase resetting”. In this process, use is made of the fact that a synchronized neuron population can be desynchronized by applying an electrical stimulus of the correct intensity and duration, provided the stimulus is applied in a vulnerable phase angle of the pathological rhythmic activity. These optimal stimulation parameters (intensity, duration and vulnerable phase) are determined as part of the calibration procedure, for example by systematically varying these parameters and comparing them with the stimulation result (e.g. attenuation of the amplitude of the band-pass-filtered feedback signal). If the telemetry device 11-13 is used, the calibration can be accelerated by using so-called phase resetting curves. Stimulation with a single high-frequency pulse train is only efficient if the stimulus is applied at the or close enough to the vulnerable phase of the activity to be stimulated. As an alternative, complex forms of stimulation can also be used. These are composed of a resetting stimulus (controlling, for example, restarting, the dynamics of the neuron population to be stimulated) and a desynchronizing high-frequency pulse train. A resetting stimulus is, for example, a short high-frequency pulse train. The advantage of this more complex method is that the complex forms of stimulation produce desynchronization independently of the dynamic state of the neuron population to be stimulated.

If a single short high-frequency pulse train is used, the control unit (4) must calculate the time when the vulnerable phase occurs in advance by means of standard prediction algorithms implemented by the electronics (control unit (4)) in order to hit it precisely enough when the threshold value determined by the calibration is exceeded. In the application of the complex stimuli according to the invention, the control unit (4) only needs to produce a new complex stimulus of the same type when the threshold value determined by the calibration is exceeded.

In the text which follows, the operation of the device according to the invention, and the treatment method, are to be explained.

According to the invention, at least one component of the group of stimulus patterns a) to d) of simple stimuli and/or complex stimuli can be used:
a) Stimulation with a short high-frequency pulse train.
b) Stimulation with a resetting, short high-frequency pulse train followed by a desynchronizing short high-frequency pulse train,
c) Stimulation with a resetting low-frequency sequence of short high-frequency pulse trains followed by a desynchronizing high-frequency pulse train.
d) Stimulation with a resetting single pulse followed by a desynchronizing short high-frequency pulse train. In this context, stimulus pattern a) is a simple stimulus and stimulus patterns c)-d) are complex stimuli.

A short high-frequency pulse train in the sense of the invention is understood to be a short high-frequency sequence of single electrical stimuli.

Short means that this sequence consists of at least 2, preferably 3, 4, 5, 6, 7, 8, 9, 10, 11, 12, 13, 14, 15, 16, 17, 18, 19, 20, 50 or up to 100 single stimuli.

All high-frequency pulse trains preferably have the same number of single stimuli. However, at least two high-frequency pulse trains can also consist of a different number of single stimuli.

The number of single stimuli of which a resetting high-frequency pulse train consists lies within the range of 2, preferably 3, 4, 5, 6, 7, 8, 9, 10, 11, 12, 13, 14, 15, 16, 17, 18, 19, 20, 50 or up to 100 single stimuli.

The number of single stimuli of which a resetting high-frequency pulse train consists preferably lies within the range from 4 to 20 single stimuli.

The number of single stimuli of which a desynchronizing high-frequency pulse train consists lies in the range of 2, preferably 3, 4, 5, 6, 7, 8, 9, 10, 11, 12, 13, 14, 15, 16, 17, 18, 19, 20, 50 or up to 100 single stimuli. The number of single stimuli of which a desynchronizing high-frequency pulse train consists preferably lies within the range from 3 to 15 single stimuli.

In the sense of the invention, high-frequency means that the frequency is preferably between 50 to 250 Hz, preferably between 80 and 150 Hz and particularly preferably between 100 and 140.

All high-frequency pulse trains preferably have the same frequency. However, at least two high-frequency pulse trains can also consist of single stimuli of different frequency.

The duration of a short high-frequency pulse train in time has a natural limit due to the fact that the short high-frequency pulse train should preferably not exceed the length of the period of the pathological neural oscillation in order to be effective. In this extent, the values specified are not restricting.

A single electrical stimulus is understood to be an electrical stimulus with essentially neutral charge, known to the expert.

Charge neutrality in the sense of the invention means that the time integral of the charge entry is essentially zero.

The time variateion of the charge entry can be symmetric or asymmetric. That is to say, in the case of these biphase single pulses, the cathodic and anodic part of the single pulse can be symmetric or asymmetric. In the symmetric case, the cathodic and the anodic part of the single pulse are identical apart from the sign of the current flow.

The amplitude of the high-frequency pulse trains can be of an order of magnitude from 0 to 16 V. The amplitude of the high-frequency pulse trains is preferably between 2 and 7 V. The usual resistance of electrode and brain tissue lies, for example, in the range from 800 to 1200Ω.

The amplitude is preferably equal for all high-frequency pulse trains but can also be different for at least two high-frequency pulse trains.

The resetting high-frequency pulse trains are preferably stronger in comparison with the desynchronizing high-frequency pulse trains. This means that in the case of the resetting high-frequency pulse trains, the amplitude and/or the number of the single pulses is greater than in the case of a desynchronizing high-frequency pulse train.

The amplitude of the single stimuli of which a resetting high-frequency pulse train consists lies in the range from 0 to 16 V, preferably between 3 and 7 V.

The amplitude of the single stimuli of which a desynchronizing high-frequency pulse train consists lies in the range from 0 to 15 V, preferably between 2 and 6 V.

A high-frequency pulse train can consist of single stimuli which preferably have the same amplitude and/or the same duration. However, at least two single stimuli can also have the same amplitude and/or the same duration.

A high-frequency pulse train can also consist of single stimuli of which at least two single stimuli have a different amplitude and/or different duration. The duration and/or the amplitude of the single stimuli can be given by deterministic and/or stochastic rules and/or combinations of the two. A combination of stochastic and deterministic rules is a functional relationship in which deterministic and stochastic terms are functionally joined to one another, e.g. by addition or multiplication. For example, the amplitude of the jth single pulse can be given by f(j), where f is a deterministic function and/or a stochastic process and/or a combination of the two.

Analogously, in the text which follows, a combination of deterministic and stochastic rules is understood to be a functional relationship in which deterministic and stochastic terms are functionally joined to one another, e.g. by addition and/or multiplication.

A low-frequency sequence of short high-frequency pulse trains preferably comprises 2-30, particularly preferably 2-20 or 2-10 high-frequency pulse trains.

The low-frequency sequence of short high-frequency pulse trains preferably consists of a periodic sequence of short high-frequency pulse trains, the frequency of which essentially corresponds to the pathological frequency—for example approx. 5 Hz in the case of Parkinson's disease.

A low-frequency sequence of short high-frequency pulse trains preferably consists of the same high-frequency pulse trains. The high-frequency pulse trains of such a low-frequency sequence can also differ with respect to their pattern.

The pattern of a high-frequency pulse train comprises the following characteristics:

A) the number of single pulses,

B) the durations of the individual single pulses,

C) the intervals between the individual single pulses,

D) the amplitudes of the individual single pulses.

Within a low-frequency sequence of short resetting high-frequency pulse trains, the pattern can be varied deterministically and/or stochastically and/or deterministically/stochastically in combination from high-frequency pulse train to high-frequency pulse train. In particular, the frequency can be varied in the individual high-frequency pulse train within a low-frequency sequence of short high-frequency pulse trains.

In the case of a multiple application of a simple stimulus and/or of a complex stimulus, the pattern of the respective high-frequency pulse trains is preferably not varied.

However, in the case of a multiple application of a simple stimulus or of a complex stimulus, the pattern of a high-frequency pulse train can also be varied from application to application. For example, in the case of a high-frequency pulse train, the number of single stimuli and/or their amplitudes and/or their durations and/or their intervals can be varied deterministically and/or stochastically and/or deterministically/stochastically in combination from application to application in a simple and/or complex stimulus.

In the case of a multiple application of a short desynchronizing high-frequency pulse train, its pattern can thus be varied deterministically and/or stochastically and/or deterministically/stochastically in combination from application to application. In particular, the frequency of the desynchronizing high-frequency pulse train can here be varied from application to application.

Similarly, in the case of a multiple application of a short resetting high-frequency pulse train, its pattern can be varied deterministically and/or stochastically and/or deterministically/stochastically in combination, from application to application. In particular, the frequency of the desynchronizing high-frequency pulse train can here be varied from application to application.

If a short high-frequency pulse train is used for desynchronization as described under a) to d), its intensity, e.g. in the sense of the charge entry occurring per unit time, is preferably lower or less than the intensity of a short high-frequency pulse train which is used for resetting.

In the case of multiple demand-controlled application, the device according to the invention can select between the forms of stimulus described under a)-d) in accordance with stochastic and/or deterministic and/or combined stochastic/deterministic rules.

In a preferred embodiment, the device is equipped with means for the cableless transmission of data such as, for example, the measurement signals and stimulation control signals so that data transmission can take place from the patient to an external receiver, for example for the purpose of therapy monitoring and optimization. In this manner, it is possible to detect early whether the stimulation parameters used are no longer optimal. In addition, a cableless transmission of data makes it possible to access a reference database and to react early to typical changes in the stimulability in the target tissue.

According to the invention, an electronic component is provided which detects the occurrence and the disappearance of a pathological feature of the electrical signal which is measured by the sensor (3, 2) and, when the pathological feature occurs, delivers at least one pulse sequence from the group according to pattern a) to d) to the electrode (2) and switches off the stimulus pattern when the pathological feature disappears. In a preferred embodiment, it comprises a univariate data processing and/or furthermore a multi-variate and/or bivariate data processing.

The electronic component is preferably constructed in such a manner that at least one of the univariate, bivariate and multivariate data processing operates with methods of statistical physics, wherein the method of statistical physics can come from the area of stochastic phase resetting.

The device according to the invention and the electronic component according to the invention can be used in medicine, preferably in neurology and psychiatry.

For example, the following diseases can be treated: Parkinson's disease, Parkinson's syndrome, epilepsy, dystonia, compulsive diseases, Alzheimer's, depression, essential tremor, tremor in the case of multiple sclerosis, tremor as a consequence of a stroke or another tumorous tissue damage.

For this purpose, the following brain regions can be stimulated:

In the case of:

Parkinson's disease: nucleus subthalamicus, thalamus, globus pallidum, nucleus ventralis intermedius thalami.
Parkinson's syndrome: nucleus subthalamicus, thalamus, globus pallidum, nucleus ventralis intermedius thalami.
Epilepsy: focal centers, hippocampus, nucleus subthalamicus, cerebellum, thalamic core regions, nucleus caudatus.
Dystonia: globus pallidum.
Compulsive diseases: nucleus accumbens, capsula interna.
Essential tremor: thalamus, nucleus ventralis intermedius thalami.
Alzheimer's: hippocampus.
Depression: hippocampus, globus pallidum.
Tremor in the case of multiple sclerosis: nucleus ventralis intermedius thalami.

What is claimed is:

1. A device for treating patients having means for stimulating brain regions, the device comprising:
at least one electrode to stimulate a brain region,
at least one sensor to measure an electrical signal, and
control means for detecting the occurrence of a pathological feature of the electrical signal which was measured by the at least one sensor and, when the pathological feature occurs, delivers at least one component from the group of stimulus sequences (a) through (d),
(a) a short high-frequency pulse train,
(b) a short high-frequency pulse train followed by a further short high-frequency pulse train,
(c) a low-frequency sequence of short high-frequency pulse trains followed by a high-frequency pulse train, or
(d) a single pulse followed by a short high-frequency pulse train to the at least one electrode.

2. The device as claimed in claim 1, wherein the control means comprises
a control system which applies short high-frequency pulse trains which in each case comprises at least 2 to 100 single stimuli, and/or
a control system which applies high-frequency pulse trains which have a frequency of 50 to 250 Hz, and/or
a control system which applies high-frequency pulse trains of the same frequency with each high-frequency application, and/or
a control system which applies single stimuli which essentially have a neutral charge.

3. The device as claimed in claim 1 or 2, wherein the control means comprises a control system which applies high-frequency pulse trains which have an amplitude of the order of magnitude between 0 to 16 V, or a control system which applies high-frequency pulse trains which have an amplitude of the order of magnitude of between 2 to 7 V, or which applies high-frequency pulse trains of the same amplitude, or which applies high-frequency pulse trains, of which at least two high-frequency pulse trains have the same amplitude.

4. The device as claimed in claim 1 to 2, wherein the control means comprises
a control system which applies high-frequency pulse trains, wherein the resetting high-frequency pulse trains are stronger than the desynchronizing high-frequency pulse trains, or a control system which applies high-frequency pulse trains, wherein the resetting high-frequency pulse trains have a higher amplitude and/or a greater number of single pulses than the desynchronizing high-frequency pulse trains.

5. The device as claimed in claim 4, wherein the control means comprises
a control system which applies resetting high-frequency pulse trains which have an amplitude of from 0 to 16 V, or a control system which applies resetting high-frequency pulse trains which have an amplitude from 3 to 7 V, and/or
a control system which applies desynchronizing high-frequency pulse trains which have an amplitude of from 0 to 15 V, or a control system which applies desynchronizing high-frequency pulse trains which have an amplitude of from 2 to 6 V.

6. The device as claimed in claim 1 or 2, wherein the control means comprises
a control system which applies high-frequency pulse trains of single stimuli which have the same amplitude and/or the same duration, or
a control system which applies high-frequency pulse trains of single stimuli, of which at least two single stimuli have the same duration and/or the same amplitude.

7. The device as claimed in claim 1 or 2, wherein the control means comprises
a control system in which high-frequency pulse trains are applied, in which trains the duration of the single stimuli and/or the amplitude of the single stimuli and/or the intervals between the single stimuli are generated by deterministic and/or stochastic methods and/or by a combination of the two, and/or
a control system in which identical high-frequency pulse trains are used within a low-frequency sequence of high-frequency pulse trains, or a control system in which the duration of the single stimuli and/or the amplitude of the single stimuli and/or the intervals between the single stimuli are varied by deterministic and/or stochastic methods and/or a combination of the two within a low-frequency sequence of high-frequency pulse trains in the individual high-frequency pulse trains, and/or
a control system in which, in a multiple application of a stimulus consisting of a number of high-frequency pulse trains, the high-frequency pulse trains used are varied with respect to the duration of the single stimuli and/or the amplitude of the single stimuli and/or the intervals between the single stimuli by deterministic and/or stochastic methods and/or a combination of the two, and/or
a control system which generates a low-frequency sequence of 2 to 30 resetting short high-frequency pulse trains, or control system which generates a low-frequency sequence of 2 to 10 short resetting high-frequency pulse trains, and/or
a control system which, in the case of a repeated stimulus application, applies at least one component of the stimulus pattern consisting of the group of patterns (a), (b), (c) and (d), or a control system which varies the application of stimuli according to the patterns (a), (b), (c) and (d) in accordance with a stochastic and/or deterministic and/or combined stochastic/deterministic sequence, and/or
a control system which recognizes the disappearance of a pathological feature and switches off the pulse trains according to patterns (a) to (d) with the disappearance of the pathological feature, and/or
a control system with univariate data processing, and/or
a control system with multivariate and/or bivariate data processing, wherein at least one of the univariate, bivariate and multivariate data processing operates with methods of statistical physics, and, in particular, the method of statistical physics comes from the area of stochastic phase resetting, and/or
the electrode comprises at least two wires, wherein the electrode acts as pickup electrode, and/or
the sensor comprises an epicortical electrode, a depth electrode, a brain electrode, a muscle electrode, the electrode or at least one component of this group, and/or
the sensor is connected to the control means via an isolating amplifier, and/or
the electrode is connected to the control means via an isolating amplifier, and/or has means for preventing an overdriving of the isolating amplifier, wherein the means for preventing the overdriving of the isolating amplifier is a relay, a transistor or an electronic filter, and/or
the control means is telemetrically connected to the sensor, and/or
the control means has means for the DC-decoupled coupling-in of the stimuli via the electrode, wherein the means for the DC-decoupled coupling-in of the stimuli comprises an optical transmitter and an optical receiver which transmit signals to the electrode, and/or
the control means is connected to a telemetry transmitter, wherein the telemetry transmitter is connected to a telemetry receiver, and the telemetry receiver, is connected to means for displaying, processing and storing data, and the means for processing data comprises a univariate data processing, and the means for processing data comprises a multivariate and/or bivariate data processing, and wherein which the means for processing data, at least one of the univariate, bivariate and multivariate methods for data processing operates with methods of statistical physics, and the method of statistical physics comes from the area of stochastic phase resetting, and/or
the electrode and the sensor are comprised at least partially in one component.

8. An electronic component, comprising:
a sensor; and
means for detecting the occurrence of a pathological feature of an electrical signal measured by the sensor and, when the pathological feature occurs, delivers at least one component from the group of control signals for (a) through (d),
(a) a short high-frequency pulse train,
(b) a short high-frequency pulse train followed by another short high-frequency pulse train,
(c) a low-frequency sequence of short high-frequency pulse trains followed by a high-frequency pulse train,
(d) a single pulse followed by a short high-frequency pulse train.

9. The electronic component as claimed in claim 8, having the functions according to claim 2, and/or
comprising a univariate data processing, and/or
comprising a multivariate and/or bivariate data processing.

10. The electronic component as claimed in claim 9, wherein at least one of the univariate, bivariate and multivariate methods for data processing operates with methods of statistical physics.

11. The electronic component as claimed in claim 10, wherein
the method of statistical physics comes from the area of stochastic phase resetting, or
the component switches off the pulse with disappearance of the pathological feature.

12. A method for use in medicine of the device as recited in claim 1 or 2.

13. A method for use in medicine of the electronic component as recited in claim 8, 9, 10 or 11.

14. A method for treating neurological and/or psychiatric diseases in which pathologically synchronous neural activity is present, the method comprising:
applying with the occurrence of a pathological feature, at least one component from the group of stimulus patterns (a) through (d),
(a) a short high-frequency pulse train,
(b) a resetting short high-frequency pulse train followed by a further desynchronizing short high-frequency pulse train,
(c) a resetting low-frequency sequence of short high-frequency pulse trains followed by a desynchronizing high-frequency pulse train, or
(d) a resetting single pulse followed by a short desynchronizing high-frequency pulse train.

15. The method as claimed in claim 14, wherein
the stimulus sequences according to stimulus patterns (a) through (d) are switched off with disappearance of the pathological feature, and/or
the functions according to the operation of the device as recited in claim 2 are used, and/or
at least one component of the group of brain regions of the thalamic regions, nucleus subthalamicus, nucleus caudatus, nucleus ventralis intermedius thalami, nucleus accumbens, thalamus, hippocampus, focal centers, globus pallidum, cerebellum, capsula interna is activated with the stimuli, and/or
diseases from the group of Parkinson's disease, Parkinson's syndrome, epilepsy, dystonia, compulsive diseases, Alzheimer's, depression, essential tremor, tremor with multiple sclerosis, tremor as a consequence of a stroke, other tissue damage or tumorous tissue damage are treated.
